(19) United States
(12) Patent Application Publication
(10) Pub. No.: US 2006/0041272 A1
(43) Pub. Date: Feb. 23, 2006

(54) Hair-styling accessory for tightening facial features

(76) Inventor: Faiza Azzeddine, Ferney-Voltaire (FR)

Correspondence Address: STURM & FIX LLP, 206 SIXTH AVENUE, SUITE 1213, DES MOINES, IA, 50309-4076, US

(21) Appl. No.: 11/219,179

(22) Filed: Sep. 2, 2005

Related U.S. Application Data
(63) Continuation of application No. PCT/EP03/50062, filed on Mar. 14, 2003.

(30) Foreign Application Priority Data
Mar. 3, 2003 (FR) FR 03.02523

(51) Int. Cl. A61F 5/08

(52) U.S. Cl. 606/204.35

(57) ABSTRACT

A hairdressing tool for re-stretching facial features by pulling two locks of hair in an opposite direction. According to the invention, said tool comprises two tongs which are joined to a ring and are disposed opposite each other according to a same direction in order to block two locks of hair which are pulled in an opposite direction, according to said direction. Preferably, the tool comprises several pairs of opposing tongs according to a direction in order to block several pairs of locks of hair. The two locks of each pair are pulled in an opposite direction according to the direction corresponding to the arrangement of two opposing tongs.

### CROSS REFERENCE TO RELATED APPLICATIONS

This application is a Continuation application of PCT/EP2003/050062 filed Mar. 14, 2003, which claims priority of Applications No. FR03.02523 filed Mar. 3, 2003, which are included in their entirety by reference made hereto.

### BACKGROUND OF THE INVENTION

1. Field of the Invention

The invention relates to a hair-styling accessory for tightening facial features by pulling two strands of hair in opposite directions along the same course.

2. Description of the Related Art

A hair-styling accessory for tightening facial features is described, for example, in the document FR-A-2071826, published on 17 Sep. 1971. According to this document, the hair-styling accessory comprises an anchoring means which is fixed on the head either by means of hair clips or by passing a strand of hair through an opening made in the centre of the anchoring means. Tighteners are intended to anchor themselves by one end to the anchoring means and to be fixed to the skin by reversible adhesion at various points on the top of the user's forehead. The tension exerted by the tighteners between the anchoring means and the points of adhesion to the skin enable the hair-styling accessory to tighten facial features. However, the use of such tighteners makes it necessary to conceal the hair-styling accessory by wearing a wig.

A hair-styling accessory for tightening facial features by pulling two strands of hair is described, for example, in the document U.S. Pat. No. 2,509,658, published on 30 May 1950. According to this document, the hair-styling accessory comprises a small bar equipped with means for holding strands of hair which are pulled by the user, relative to the bar. The bar itself is fixed to the hair by a comb or by a pivoting tongue. With an accessory of this type, the facial features are tightened by means of the strands of hair themselves, so it is not necessary to conceal the accessory by wearing a wig. However, the design of this accessory, in which the means for holding the strands of hair relative to the bar are different from the means for holding the bar itself relative to the hair, appears complicated and does not guarantee the balancing of forces between the strands of hair used for tensioning and the strands of hair used for fixing, over time. It also appears necessary to provide a plurality of bars for tightening the facial features at a plurality of points.

A hair-styling accessory for tightening facial features by pulling two strands of hair in opposite directions along a same course is described, for example, in the document U.S. Pat. No. 2,307,714, published on 5 Jan. 1943. According to this document, the hair-styling accessory comprises two grippers for locking strands of hair and a means for exerting tension between the two grippers when they are disposed at two opposing points of the user's head, for example in the vicinity of the left and right temples. The means comprises, more particularly, a locking box which is connected to each of the two grippers by a string. The locking box enables one of the two strings to be locked in order to adjust the tension exerted between the two hair grippers. The hair grippers as well as the strings and the locking box are small in size and may be concealed in the head of hair in such a way that it is not necessary to wear a wig. The strands of hair are pulled in opposite directions in the region of the temples by strings for holding the locking box flat against the surface of the head so that the same strands of hair enable the facial features to be tightened and the box, by means of which a tension is created between the two grippers, to be held in position. However, a hair-styling accessory of this type appears to be limited to a single pair of grippers which enable two strands of hair to be pulled in opposite directions in view of the fact that it is awkward to adjust the tension of the strings by means of the locking box.

### SUMMARY OF THE INVENTION

One of the objects of the invention is to design a hair-styling accessory so as to simplify the positioning thereof by a user while maintaining a design in which the strands of hair which are pulled so as to tighten the facial features also serve to keep the grippers locked in position relative to the head of hair and in which the hair-styling accessory remains discreet so that it may be used without the need to wear a wig.

The invention accordingly relates to a hair-styling accessory for tightening facial features by pulling two strands of hair in opposite directions, characterised in that it comprises two grippers integral with a base disposed opposite to one another along the same course so as to each lock one of the two strands of hair which are pulled in opposite directions along this course while keeping the base in position on the head of hair.

The base, for example a ring, equipped with the two mutually opposed grippers simplifies the design of the hair-styling accessory according to the invention, in particular by the elimination of a locking box and of tensioning strings between the locking box and each hair gripper. The two grippers which are mutually opposed on the base enable the user to pull a strand of hair and to lock it in the corresponding gripper in a single gesture. The user therefore finds it easy and natural to use the hair-styling accessory according to the invention.

According to an advantageous embodiment of the invention, the base comprises a plurality of pairs of grippers which are opposed along a course so as to lock a plurality of pairs of strands of hair, the two strands in the same pair being pulled in opposite directions along the course of the two opposing grippers. The same hair-styling accessory according to the invention therefore enables the facial features to be tightened from different tensioning points.

Further advantages will emerge from the description of two embodiments of the invention which are illustrated in the drawings.

### BRIEF DESCRIPTION OF THE DRAWINGS

FIG. 1 is a perspective view of hair-styling accessory according to a first embodiment of the invention.

FIG. 2A is a plan view of a hair-styling accessory according to FIG. 1.

FIG. 2B shows the hair-styling accessory illustrated in FIG. 2A along a cross section AA, the grippers for locking strands of hair being in the open position.

FIG. 2C shows the hair-styling accessory illustrated in FIG. 2A along the cross section AA in FIG. 2B, the grippers for locking the strands of hair being in the closed position.

FIG. 3 shows a schematic view of a plurality of pairs of strands of hair pulled at a plurality of points of a user's head.

FIG. 4 is a perspective view of a hair-styling accessory according to a second embodiment of the invention.

### DETAILED DESCRIPTION OF THE PREFERRED EMBODIMENT

As mentioned hereinbefore, the invention relates to a hair-styling accessory for tightening the facial features by pulling two strands of hair in opposite directions.

According to a first embodiment of the invention, shown in FIGS. 1 and 2A to 2C, the accessory comprises two grippers 31A and 31B integral with a ring of calvarial shape 1 and arranged in a mutually opposed manner along the same course L3 so as to lock the two strands of hair 31 and 31′ in opposite directions along this same course L3. In this embodiment, the calvarial dome 1 is a flat disk which is a few millimetres thick and has a central opening 4 for the passage of a strand of hair and enables the ring 1 placed on the top of the head to be covered discreetly. By way of example, the calvarial dome 1 has a diameter of 40 mm and a thickness of 4 mm.

The grippers 31A and 31B used to lock the strands of hair in opposite directions along the same course comprise, as shown in FIGS. 2B and 2C, a tongue 11 which pivots relative to the calvarial dome 1 about a hinge 13 so as to pass from an open position where each strand of hair 31 or 31′ is wound round the tongue 11 to a closed position where the tongue 11 is kept closed by a claw 17 for locking the wound strand of hair.

Preferably, the calvarial dome 1 comprises a plurality of opposing pairs of grippers (51A and 51B, 71A and 71B, 91A and 91B, for locking a plurality of pairs of strands of hair in a plurality of courses, the two strands of each pair being pulled in opposite directions along the same course L5, L7 and L9 respectively.

In order to position the hair-styling accessory, according to this first embodiment, the user divides her hair into eight portions and uses two clips to hold the eight portions of hair. She then forms very fine strands of hair, using, for example, about fifteen hairs, of which the roots are approximately at a few centimetres from the skin of the face. As illustrated in FIG. 3, one strand 31 is formed for tightening the facial features from the centre of the forehead, one strand 71 is formed for exerting traction in the region of the right eyebrow, one strand 51 is formed for exerting traction in the region of the left eyebrow and one strand 91 is formed for exerting traction on each of the user's two temples.

In relation to each of the aforementioned strands 31, 71, 51 and 91, a strand is formed along the same course but in opposite directions relative to the top of the head. Therefore, one strand 31′ is formed at the back of the head in the opposite direction to and along the same course as the strand 31, of which the root is in the vicinity of the centre of the forehead. Similarly, one strand 71′ is formed at the back left of the head in the opposite direction to and along the same course as the strand 71 which has its root in the region of the right eyebrow and one strand 51′ is formed at the back right of the head in the opposite direction to and along the same course as the strand 51 which has its root in the region of the left eyebrow.

The user then arranges the hair-styling accessory at the top of her head and begins winding the strands of hair 31 and 31′ which issue from the centre of the forehead and the back of the head, around the tongue 11 in the open position of the respective grippers 31B and 31A arranged in a mutually opposed manner on the ring 1 along the course L3. The user obviously adjusts the tension which she feels on her forehead and on the back of her head by pulling the corresponding strands of hair 31 and 31′ to a greater or lesser extent so as to lock them by folding the tongue 11 down into the claw 17 of the grippers 31A and 31B. The user continues in the same way with the strands 71 and 71′ issuing from the right eyebrow and the back left of the head, then with the strands 51 and 51′ issuing from the left eyebrow and the back right of the head and with the strands 91 and 91′ issuing from the user's two temples.

It should be noted that if the user winds a strand of hair 31 or 31′ once round the tongue 11 of the locking grippers 3A and 3B, she can then adjust the tensions exerted between the two opposing grippers by pulling the free edge of the strand locked by either of the grippers in order to increase the tension along the corresponding course L3.

It appears that the hair-styling accessory according to this first embodiment of the invention is simple and natural to use as two strands 31A and 31B pulled by the user in opposite directions along the same course L3 and locked by two opposing grippers integral with the ring allow the facial features to be tightened and the ring to be held in a stable position on the head of hair at the top of the user's head. Furthermore, the plurality of opposing pairs of grippers enables the facial features to be tightened by pulling strands which have their roots at a plurality of points of the face, for example in the region of the temples and of the eyebrows and of the centre of the forehead. The arrangement of the calvarial dome at the top of the head and its small size enable the hair-styling accessory to be concealed easily in the user's head of hair without necessitating the wearing of a wig.

The user removes the hair-styling accessory in an equally simple and natural manner by opening the locking grippers one by one to release the strands of hair.

A second embodiment of the invention illustrated in FIG. 4 differs from the preceding embodiment in that the grippers 32A and 32B for locking the strands of hair comprise slideways 21A or 21B which guide a clamping member 23A or 23B having a shape which is complementary with the slideways in order to wedge the strands of hair in a narrowed portion 25A or 25B of the slideway 21A or 21B.

Preferably, the clamping member 23A or 23B is held inside the slideway by a stud 27A or 27B which is integral with the slideway 21A or 21B inserted in an aperture 29A or 29B in the clamping member. According to this embodiment, the user releases the clamping member from the narrowed portion of the slideway in order to wind a strand of hair 32 or 32′ round the clamping member and between this clamping member and the slideway. The user removes the strand of hair through the narrowed portion of the slideway and then slides the clamping member into this narrowed portion in order to lock the strand of hair between the clamping member and the slideway.

In the first or second embodiment of the invention, the calvarial dome 1 or 2 is preferably a single-piece member made of injection-moulded plastics material. By way of example, a polypropylene or any other thermoplastic material is used by heating it then injecting it into a mould under elevated pressure. Preferably, the clamping members of the second embodiment are also members made of injection-moulded plastics material and, according to the first embodiment, the calvarial dome 1 as well as the various opposing pairs of grippers form a single one-piece member made of injection-moulded plastics material. In the case illustrated, the hinge 13 of the tongue 11 is formed by an attenuation of plastics material.

It is provided to replace the annular base with a rectangular, triangular, oval or cross-shaped plate.

An accessory according to the invention may be used with various forms of hair style such as long hair, tied-up hair, hair worn with or without slight backcombing, for better concealing the ring if necessary. The ease of positioning of the hair-styling accessory enables the facial features to be tightened in an instantaneous, temporary and occasional manner. It thus allows various effects of ageing of the face, such as wrinkles round the eyes and on the forehead, slackening of the facial muscles, etc. to be masked.

1. Hair-styling accessory for tightening facial features by pulling two strands of hair on a head of hair in opposite directions, comprising two grippers integral with a base disposed opposite to one another along the same course so as to lock the two strands of hair which are pulled in opposite directions along the course while keeping the base in position on the head of hair.

2. Hair-styling accessory according to claim 1, wherein the base comprises a plurality of pairs of grippers which are opposed along a course so as to lock a plurality of pairs of strands of hair, the two strands of each pair being pulled in opposite directions along the course corresponding to the arrangement of two opposing grippers.

3. Hair-styling accessory according to claim 1 wherein the grippers comprise tongues which pivot relative to the base about a hinge between an open position enabling the strands of hair to be wound round the hinge and a closed position for locking the strands of hair.

4. Hair-styling accessory according to claim 3, wherein the tongue is held in the closed position by a claw integral with the base.

5. Hair-styling accessory according to claim 1, wherein the grippers comprise slideways which guide a clamping member of complementary shape so as to wedge the strands of hair in a narrowed portion of the slideway.

6. Hair-styling accessory according to claim 5, wherein the clamping member is held inside the slideway by a stud which is inserted in an aperture in the clamping member.

7. Hair-styling accessory according to claim 1, wherein the base and the grippers are made of injection-moulded plastics material.

8. Hair-styling accessory according to claim 7, wherein the base and the grippers form a single-piece member made of injection-moulded plastics material.

9. Hair-styling accessory according to claim 1, wherein the base is annular, rectangular, triangular, oval or cross-shaped.

10. Hair-styling accessory according to claim 2 wherein the grippers comprise tongues which pivot relative to the base about a hinge between an open position enabling the strands of hair to be wound round the hinge and a closed position for locking the strands of hair.

11. Hair-styling accessory according to claim 10, wherein the tongue is held in the closed position by a claw integral with the base.

12. Hair-styling accessory according to claim 2 wherein the grippers comprise slideways which guide a clamping member of complementary shape so as to wedge the strands of hair in a narrowed portion of the slideway.

13. Hair-styling accessory according to claim 12, wherein the clamping member is held inside the slideway by a stud which is inserted in an aperture in the clamping member.

14. Hair-styling accessory according to claim 2, wherein the base and the grippers are made of injection-moulded plastics material.

15. Hair-styling accessory according to claim 3, wherein the base and the grippers are made of injection-moulded plastics material.

16. Hair-styling accessory according to claim 4, wherein the base and the grippers are made of injection-moulded plastics material.

17. Hair-styling accessory according to claim 5, wherein the base and the grippers are made of injection-moulded plastics material.

18. Hair-styling accessory according to claim 6, wherein the base and the grippers are made of injection-moulded plastics material.

19. Hair-styling accessory according to claim 14, wherein the base and the grippers form a single-piece member made of injection-moulded plastics material.

20. Hair-styling accessory according to claim 2, wherein the base is annular, rectangular, triangular, oval or cross-shaped.
